(19) United States
(12) Patent Application Publication
(10) Pub. No.: US 2021/0140949 A1
(43) Pub. Date: May 13, 2021

(54) METHOD FOR DETERMINING ENDOGENOUSLY FORMED CARBONYL COMPOUNDS

(71) Applicant: THANARES GMBH, Potsdam (DE)

(72) Inventors: Hans-Michael THIEDE, Postdam (DE); Wolfgang KEHR, Berlin (DE)

(21) Appl. No.: 17/050,340

(22) PCT Filed: Apr. 16, 2019
(86) PCT No.: PCT/DE2019/000106

§ 371 Date: Oct. 23, 2020

(30) Foreign Application Priority Data
Apr. 25, 2018 (DE) 10 2018 003 367.9

(51) Int. Cl. G01N 33/52; C12Q 1/04

(52) U.S. Cl. CPC G01N 33/52; C12Q 1/04

(57) ABSTRACT

The invention relates to a method for determining a condensation product of a Pictet-Spengler reaction between a carbonyl compound, in particular an aldehyde, preferably formaldehyde, and an amine in a body fluid removed from a human and/or in cells taken from a human, wherein a chemical, physical, or physico-chemical determination of the condensation product as an analyte is carried out on the body fluid and/or the cells. The invention also relates to novel analytes useful therefor.

### FIELD OF THE INVENTION

The invention relates to a method for qualitatively, semi-quantitatively, or quantitatively determining aldehyde-producing microorganisms in the intestinal tract of a human on a body fluid removed from a human and/or on cells taken from a human.

Prior art and background of the invention

M. Alzheimer and M. Parkinson are the two neurodegenerative diseases in old age. The etiology of both diseases is still unclear, and a curative therapy is not possible.

Since more than 10 years, it is discussed whether Parkinson's disease starts in the intestines (Braak H, Del Tredici K, Rüb U, de Vos RAI, Jansen Steur ENH, Braak E (2003) Staging of brain pathology related to sporadic Parkinson's disease. Neurobiol. Aging 24:197-211). Among many other factors, modified microbiota (dysbiosis) and neurotoxins created therefrom were mentioned as factors. To the intestinal metabolites created by the microbiota having a high responsiveness belong carbonyl compounds, e.g., formaldehyde, acetaldehyde, and methylglyoxal.

The aldehydes formed in the metabolism of eukaryotes and prokaryotes are very responsive molecules. In particular, formaldehyde is a very responsive molecule that is formed in considerable amounts in the metabolism from methanol in many organs of the human organism, and due to its short half-time (˜-2 min), it can also be detected in the blood in a relatively low concentration (˜2-3 μg/ml). Formaldehyde is converted by the enzyme aldehyde dehydrogenase to formic acid.

In addition to the carcinogenic and mutagenic effect, formaldehyde promotes the aggregation of proteins and nucleic acids, inter alia, the aggregation of the tau protein that plays an important role in the axonal transport in nerve cells. These amyloid-like aggregates of the tau protein are neurotoxic, and an accumulation of these aggregates will lead to a degeneration and finally to the death of the nerve cells (Nie CL, Wei Y, Chen X, Liu YY, Dui W, Liu Y, Davies MC, Tendier SJ, He RG. PLoS One.

2007 Jul 18; 2(7)). Tau protein aggregate and beta amyloid depositions, another protein aggregate generated by formaldehyde, are found in the brain of patients with Alzheimer's disease and correlate with the disease. Increased exposure of the brain to formaldehyde will lead to a significant deterioration of the memory (Tong Z, Han C, Luo W, Li H, Luo H, Qiang M, Su T, Wu B, Liu Y, Yang X, Wan Y, Cui D, He R. Sci. Rep. 2013; 3:1807) and other cognitive brain functions (He R, Lu J, Miao J. Sci. China Life Sci. 2010 Dec; 53(12):1399-404; Lu J, Miao J, Su T,

Liu Y, He R. Biochim. Biophys. Acta. 2013 August; 1830(8):4102-16).

In clinical studies, a higher formaldehyde concentration was measured in the urine of Alzheimer patients, depending on the degree of the dementia, (Tong Z,

Zhang J, Luo W, Wang W, Li F, Li H, Luo H, Lu J, Zhou J, Wan Y, He R. Neurobiol. Aging. 2011 Jan; 32(1):31-41). Concerning the reason of this higher formaldehyde excretion of Alzheimer patients, there exist no studies up to now. In a new publication about the measurement of formaldehyde in brain tissue (Yue X, Zhang Y, Xing W, Chen Y, Mu C, Miao Z, Ge P, Li T, He R, Tong Z. Anal. Cell Pathol. (Amst). 2017; 2017:9043134), it could be shown that, after systemic formaldehyde administration, the concentration of formaldehyde in the brain increases.

It is known that, beside the generation of formaldehyde in the metabolism by enzymatic demethylization and oxidative desamination, processes that with regard to a decrease of the formaldehyde loading are only possible with pharmacological interventions having side effects, the formaldehyde production in the large intestine by bacteria and fungi is of great importance. Already in 1924, it could be shown that formaldehyde is formed in bacteria cultures.

Methylglyoxal is a so-called dicarbonyl and is formed during the degradation of glucose and fructose. Furthermore, the occurrence during the protein and lipid degradation has also been described. An increased production with diabetes mellitus is known and is held responsible for the development of typical diabetes complications, e.g., vessel damages, neuropathies and the formation and accumulation of AGE (advanced glycation end products) (Mukohda M, Okada M, Hara Y, Yamawaki H. J. Pharmacol. Sci. 2012; 118(3):303-10; Thornalley P J, Langborg A, Minhas H S. Biochem. J. 1999 Nov 15;344 Pt 1:109-16). The production of methylglyoxal by bacteria isolated from human feces has been documented in 1989 by Baskaran et al. (Baskaran S, Rajan DP, Balasubramanian KA. J. Med. Miorobiol. 1989 Mar.; 28 (3):211-5).

The highest number of germs is found to be 10¹¹-10¹³ in the colon. The small intestine normally has a low colonization only, and the number of germs is about <10³ c.f.u./ml (number of colony-forming germs per ml jejunal aspirate) for healthy humans. If, however, an increased colonization of the small intestine with germs occurs, then this is called SIBO (small intestinal bacterial overgrowth) or also SIFO (small intestinal fungal overgrowth), wherein according to definition the number of colony-forming germs in the jejunal aspirate is found to be >10³ c.f.u./ml. It is irrelevant, here, which species of microbiota are involved (Rezaie A, Buresi M, Lembo A, Lin H, MoCallum R, Rao S, Schmuison M, Valdovinos M, Zakko S, Pimentei M. Am. J. Gastroenterol. 2017 May; 112(5):775-784).

The breath tests currently used in SIBO diagnostics with different sugars (glucose, lactulose, fructose) have incorrect results and cannot replace the invasive technique of obtaining aspirate from the small intestine with subsequent microbiological investigation for diagnostic confirmation.

Meanwhile, various “risk factors” are known that lead to a higher SIBO incidence. To these belong disorders of the intestinal motility leading to a prolonged orocecal transit time (e.g., hypothyreosis). Further risk factors are age and a defective ileocecal valve function. In the long-term therapy with PPI (proton-pump inhibitors), diabetes mellitus, M. Parkinson, restless legs, systemic sclerosis, cystic fibrosis, chronic pancreatitis, celiac disease and gastroparesis, a SIBO can also frequently be detected.

The Sibo incidence in Parkinson patients is high and is about 40 to 60% (Fasano A, Bove F, Gabrielii M, Petraooa M, Z0000 MA, Ragazzoni E, Barbaro F, Piano C, Fortuna S, Tortora A, Di Giaoopo R, Campanale M, Gigante G, Lauritano E C, Navarra P, Marooni S, Gasbarrini A, Bentivogiio A R. Mov. Disord. 2013 Aug; 28(9):1241-9). After eradication, the relapse rate is high again after half a year already and is stated to be 50%. The occurrence of formaldehyde-producing microbiota in the upper small intestine sections, in a non-physiological amount and possibly also composition, permits the possibility of a condensation reaction with L-dopa delivered during the Parkinson therapy. The condensation products created in the intestine are resorbed and are thus systemically available.

L-dopa is, thus, a formaldehyde scavenger, however, the L-dopa bioavailability is reduced, and next to nothing is known about the pharmacology and toxicology of the created condensation products.

In a mouse model of Alzheimer's disease, it was shown that in the brain of mice without intestinal bacteria, the depositions typical for M. Alzheimer were drastically reduced compared to mice with intact microbiota (Harach T, Marungruang N, Duthilleul N, Cheatham V, McCoy KD, Frisoni G, Neher JJ, Fak F, Jucker M, Lasser T, Boimont T. Sci. Rep. 2017 Feb 8; 7:41802).

From these results, it can be derived that by reduction of formaldehyde-forming germs in the intestinal tract, the risk of the occurrence of neurotoxic protein aggregates and corresponding dementia diseases typical for M. Alzheimer can significantly be reduced.

A requirement for such preventive measures is the development of a reliable test for the detection and quantitative determination of formaldehyde in the intestinal tract. Such a test does currently not exist, and the intestinal formaldehyde production cannot be diagnosed up to today with regard to a quantitative reaction mixture. Due to the extremely high responsiveness of formaldehyde and the thus resulting short biological half-time, the determination of free and reversibly bound formaldehyde, e.g., in the blood, is no reliable measure for the amount or concentration of formaldehyde in vivo.

### Technical Object of the Invention

The invention is based on the technical object to close a diagnostic gap with regard to the determination of formaldehyde endogenously formed by microorganisms in an intestinal tract of a human in removed body fluids or cells.

Basics of the invention and preferred embodiments

For the solution of this technical object, the invention teaches a method for the qualitative, semi-quantitative, or quantitative determination of aldehyde-producing microorganisms in the intestinal tract, in particular of the small intestine, of a human on a body fluid removed from a human and/or on cells taken from a human, wherein on the body fluid and/or the cells a qualitative, semi-quantitative, or quantitative chemical, physical, or physico-chemical determination of a condensation product of a Pictet-Spengler reaction between a carbonyl compound, in particular an aldehyde, preferably formaldehyde, and an amine is carried out.

The invention also relates to a method for the qualitative, semi-quantitative, or quantitative determination of a condensation product of a Pictet-Spengler reaction between a carbonyl compound, in particular an aldehyde, preferably formaldehyde, and an amine in a body fluid removed from a human and/or in cells taken from a human, wherein in the body fluid and/or the cells a qualitative, semi-quantitative, or quantitative chemical, physical, or physico-chemical determination of the condensation product is carried out.

A qualitative analysis detects the presence or the absence of an analyte. An analyte is absent, if its concentration is below the detection limit of the employed analysis method. A semi-quantitative analysis is a determination, whether the amount of the analyte to be determined is within certain amount ranges. For this purpose, the amount ranges are quantitatively defined and form coarse patterns, to which definitions, such as slightly/moderately/clearly/strongly increased (or reduced) are assigned. A quantitative analysis provides an indication of a quantity (or an indication of a concentration) with an accuracy that is equivalent to the accuracy of measurement of the employed method.

For the chemical, physical, or physico-chemical determination of the condensation product of the Pictet-Spengler reaction between a carbonyl compound, in particular an aldehyde, preferably formaldehyde, and an amine, basically all methods are suitable that the person skilled in the art may rate as suitable. Only as examples, as physical methods, mass spectrometry (if applicable, as an GC-MS combination) or NMR are mentioned. A physico-chemical method is, for instance, HPLC. As a chemical method, for instance, the derivative method may be mentioned.

The invention is based on that it is known from other contexts, the synthesis of organic compounds, that in the Pictet-Spengler synthesis of heterocycles, already under mild conditions in a reaction between an amine and an aldehyde under water elimination, a condensation product will be created.

It was found that, in addition to amines, aromatic amino acids such as, e.g., L-dopa and L-dops with formaldehyde react under mild conditions (already at room temperature) in an analogous manner, whereby the 3-carboxy heterocycles are formed. Formaldehyde forms, with a series of substances available in a body or supplied to the body, analogous condensation products, which are sufficiently stable, the essential metabolism of which is known and which can be measured in body fluids such as blood, urine, and brain fluid.

In the following, as an example, the reaction of L-dopa with formaldehyde is shown:

[chemical structure]

It is preferred if the microorganisms to be determined are carbonyl compounds-producing, preferably aldehyde-producing, in particular formaldehyde-producing.

The body fluid may be selected from the group consisting of urine, full blood, blood plasma, serum, and liquor. The cells may be selected from the group consisting of erythrocytes, leukocytes, and thrombocytes.

The amine is, for instance, a catecholamine with a primary amine group, in particular dopamine or L-dopa.

The amine may in particular be a substance of formula I (the definitions of the residues identically apply for formula I and formula II):

[chemical structure]

wherein Ri is selected from —CH₃, —CH₂OH, —COOH, —COOCH₃, —COOC2H₅, —CONH₂, —OH, —OCH₃, —OC₂H₅, wherein R₂, R₃ and R₄, each independently of the other, is selected from —H, —OH, —CH₃, —C21-15, —OCH₃, —OCH₃, —OC₂H₅,
wherein R₁ and R₂, alternatively to the above definitions, may together also be ═O,
wherein Rs is selected from —H, —CH₃ and —C₂H₅,
wherein R₆ is —H or —OH,
wherein R₇ is —H or —OH,
wherein R₈ and R₉, each independently of the other, are selected from —H, —OH, —OCH₃ and —OC₂H₅, and
wherein R₁₀ is —H or —OH.

The condensation product preferably is 6,7-dihydroxy-3-carboxy-1,2,3,4-tetrahydroisoquinoline or 7,8-dihydroxy-3-carboxy-1,2,3,4-tetrahydroisoquinoline.

The invention further, and independently of the suitability in a method described above, teaches novel compounds, namely 7,8-dihydroxy-1,2,3,4-tetrahydroisoquinoline or a 7,8-dihydroxy-1,2,3,4-tetrahydroisoquinoline derivative, wherein in the 7,8-dihydroxy-1,2,3,4-tetrahydroisoquinoline derivative, —H in position 3 may be replaced by —CH₃, —CH₂OH, —COOH, —COOCH₃, —COOC2H₅, —CONH₂, —OH, —OCH₃, —OC2H₅, —OH, —OCH₃, or —OC₂H₅, one —H or both —H in position 4, independently of the other, identically or differently, may be replaced by —OH, —CH₃, —C2H₅, —OCH₃, or —OC₂H₅, and one —H or both —H in position 5 or 6, independently of the other, identically or differently, may be replaced by —OH.

The invention finally teaches the use of 7,8-dihydroxy-1,2,3,4-tetrahydroisoquinoline or of a 7,8-dihydroxy-1,2,3,4-tetrahydroisoquinoline derivative as described above or of 6,7-dihydroxy-3-carboxy-1,2,3,4-tetrahydroisoquinoline or of a 6,7-dihydroxy-3-carboxy-1,2,3,4-tetrahydroisoquinoline derivative, wherein —H in position 3 may be replaced by —CH₃, —CH₂OH, —COOH, —COOCH₃, —CO0C2H₅, —CONH₂, —OH, —OCH₃, —OC₂H₅, —OH, —OCH₃, or —OC₂H₅, one —H or both —H in position 4, independently of the other, identically or differently, may be replaced by —OH, —CH₃, —C₂H₅, —OCH₃, or —OC2H₅, one —H or both —H in position 7 or 8, independently of the other, identically or differently, may be replaced by —OH, as an analyte in a method according to the invention.

In the following, the invention is explained in more detail with reference to examples.

### EXAMPLE 1

### Reaction: dopamine+formaldehyde, Sample Collection

In this example, as a reactant for the carbonyl compound, an amine with a primary amine group is first supplied to the body. The formulation of reactants (e.g., dopamine) may be made in all possible pharmaceutical forms for oral application. Here are also included, however, reactants that are comprised in food (e.g., dopamine in bananas and banana peels).

Standardized dosages of a reactant are administered orally, and the condensation products developed in the intestinal tract (and/or its known endogenous metabolites) with formaldehyde are analyzed after predetermined times in urine or blood, respectively.

To the plant genus Musa belongs the generally known banana, a berry fruit, of which various types exist. In the fruit pulp and in particular in the peel can be found high contents of dopamine (100-500 mg/100 g). These indications in the literature could be confirmed with own measurements. A preparation of an extract from banana peels with a defined amount of dopamine can be achieved as follows.

100 grams banana peel (bio-quality) are cut into approximately 2×2 cm large pieces and are added to a 1-1 beaker. After addition of 350 ml of 0.1 N hydrochloric acid, the peel pieces are cooked on the heating plate for 15 minutes, then homogenized with a mixer. An aliquot is centrifuged in the Eppendorf vial, and the amount of dopamine in the total volume is measured with HPLC. The homogenate is then filtered, and at least 200 ml of filtrate should be obtained from the total amount of homogenate. The filtrate is adjusted with NaOH to approximately pH 5, the amount of dopamine is again determined, and diluted with water until approximately 100 mg of dopamine are obtained in 250 ml.

250 ml of the diluted filtrate are brought to cooking, and 70 grams of oat flakes are added, again cooked, and left for 20 minutes at room temperature. The test meal is now ready for intake.

Urine collection periods after intake: 0-12, 12-24, 24-36, and 36-48 hours. Urine collection occurs in collection containers prepared with a preservation solution. After every urine collection period, the urine volume is measured, an aliquot (2×10 ml) is separated and frozen at −20° C. at least.

The determination of norsalsolinol and dopamine (free and after thermal hydrolysis) is made with HPLC and electrochemical detection.

### EXAMPLE 2

### Clinical, Diagnostic and Therapeutic Relevance of the detection of endogenous condensation products of L-dopa, alpha-methyl-dopa and droxidopa with formaldehyde

Parkinson's disease is characterized by a progressing degeneration of dopamine-containing nerve cells of the central nervous system and of the intestinal tract (ENS) and the associated occurrence of the symptoms lack of motion, muscle rigidity, and tremor. In addition to the classic triad, there are a series of symptoms such as obstipation, sleep disturbances, and loss of the sense of smell that can precede the occurrence of the motoric problems by years and are explained as an autonomic dysfunction (Siderowf A, Stern MB. Ann. Neurol. 2008 December;

64 Suppl. 2:S139-47). In newer concepts of the pathogenesis of M. Parkinson, a closer correlation between a modified composition of the microbiota of the intestine and the occurrence of the disease is deemed possible (Bedarf J R, Hildebrand F, Coelho L P, Sunagawa S, Bahram M, Goeser F, Bork P, Wüllner U. Genome Med. 2017 Apr. 28; 9(1):39). The protein aggregates typical for the disease should be possible through the so-called “spreading” from the ENS of the intestine via the N. vagus. Further, the hypothesis postulated by Braak (supra) with regard to the development of M. Alzheimer also considers the ENS as the location of the development of the disease.

An essential principle of the medicamentous Parkinson therapy is the compensation of this dopamine deficiency. The therapy with L-dopa was an enormous breakthrough in the Parkinson therapy at the beginning of the sixties and is still today one of the most effective drugs. L-dopa passes, other than dopamine, the blood-brain barrier, and is quickly converted to dopamine in the brain. In order that as little L-dopa as possible is degraded already before reaching the brain, L-dopa is always administered in combination with a decarboxylase inhibitor (benserazid or carbidopa). Often, a catechol-O-methyltransferase inhibitor (entacapon or tolcapon) is further added, in order to inhibit the degradation of L-dopa and dopamine.

The symptoms of Parkinson's disease can satisfactorily be treated with L-dopa, the degeneration of the dopaminergic nerve cells, however, is not delayed by the therapy. There are even indications that the neurodegeneration will more quickly progress with the L-dopa treatment.

### EXAMPLE 3

### L-Dopa Determination

In the last years, different/various methods for the

L-dopa determination in plasma/serum, organs, and urine have been developed and published (Thiede HM, Kehr W. Naunyn Sohmiedebergs Arch. Pharmacol. 1981 Dec; 318(1):19-28; Lee M, Nohta H, Ohtsubo K, Yoo B, Ohkura Y. Chem. Pharm. Bull. (Tokyo). 1987. January; 35(1):235-40).

Due to the low endogenous L-dopa concentration, mainly electrochemical detection methods (coulometric/amperometric), in conjunction with an HPLC method, offer the required sensitivity (Ishimitsu T, Hirose S. Anal. Biochem. 1985 Nov. 1; 150(2):300-8. Blandini F, Martignoni

E, Pacchetti C, Desideri S, Rivellini D, Nappi G. J. Chromatogr. B. Biomed. Sci. Appl. 1997 Oct. 24; 700(1-2):278-82).

As separating materials in the HPLC, so-called reverse phases were and are employed, which, in conjunction with ion pair reagents and an organic modifier, permit a separation of the substances of polar compounds having similar structures. When attempting to detect, with short HPLC retention times, as many compounds as possible, there is a risk that the chromatographic system is over-challenged and so-called coelutions will result, with compounds unknown up to now remaining “undetected”.

Since a couple of years, new separating materials are also offered on the market, by means of which even in purely aqueous media/buffer solutions excellent separations of substances are possible (Triart C18 of YMC).

### EXAMPLE 4

### Structural Analysis of Potential Analytes

In a development of novel methods for determining L-dopa in human urine samples, anonymized 24-hours urine collection samples of Parkinson patients treated with L-dopa were available. In the HPLC chromatograms of the treated Parkinson patients investigated, there appeared, in addition to the L-dopa signal, further signals that could not be assigned to any of the known endogenous compounds. Surprising was also the strength of the signal in the chromatogram, due to which it seemed reasonable to characterize the new unknown substances.

The kind of the selected sample preparation (cation exchanger with elution at the isoelectric point, subsequent binding of aluminum oxide in the cation exchanger eluate with subsequent elution in acid condition) enabled structural standards significantly reducing the list of the substances coming into question. Considerations led to the assumption that these could be dihydroxy-tetrahydroisoquinolines from the reaction of formaldehyde with L-dopa not yet described in the literature.

In the following, selected aromatic amino acids (L-dopa, alpha-methyldopa, DOPS, m-tyrosine, 5-HTP) were reacted with various simple aldehydes, and the products generated in the Pictet-Spengler reaction were isolated.

The reaction of the reactants occurred with a 5-fold molar excess of aldehyde in sodium phosphate buffer pH 6.5 or ammonium acetate buffer pH 6.5, each 0.1 M. Among the aldehydes investigated, formaldehyde showed the largest reactivity with all reactants, and at room temperature already a complete conversion was achieved. In the subsequent measurement of the reaction products with HPLC and coulometric detection, all aromatic amino acid condensation products exhibited two signals. NMR investigations of the isolated compounds yielded that in the reaction of L-dopa with formaldehyde, approximately 90% of 6,7-dihydroxy-3-carboxy-1,2,3,4-tetrahydroisoquinolines (3-CNSa) and approximately 10% of 7,8-dihydroxy-3-carboxy-1,2,3-tetrahydroisoquinolines (3-CNSb) were obtained. For the remaining reactants with a catechol structure, the 6,7- or 7,8-dihydroxy structures, respectively, were also formed. While the 6,7-dihydroxy-3-carboxy-1,2,3,4-tetrahydroisoquinolines as substances are known and described, there exists no information about the 7,8-dihydroxy-3-carboxy-1,2,3-tetrahydroisoquinolines, they are unknown as substances.

### EXAMPLE 5

### Effects of the Condensation Reaction of L-Dopa and formaldehyde to 3-CNSa and 3-CNSb

Due to the condensation reaction, the availability of L-dopa in the organism decreases, i.e. less L-dopa is available for the conversion into dopamine in the brain. The efficiency of the therapy is thus reduced.

According to studies of Kurnik et al. (Kurnik M, Gil K, Gajda M, Thor P, Bugajski A. Folia Histochem. Cytobiol. 2015; 53(1):49-61), salsolinol stimulates the formation of alpha-synuclein. Alpha-synuclein is a protein, to which is attributed a toxic effect for certain nerve cells, mainly, however, for dopaminergic neurons of the Substantia nigra, where it, in the form of protofibrils, is regarded as a contributory cause of oxidative stresses and the neuronal cell death resulting therefrom. Alpha-synuclein accumulates in dopaminergic nerve cells of Parkinson patients and is regarded as a marker for the disease.

N-methyl-salsolinol causes an apoptosis induction with subsequent neurodegeneration of dopaminergic nerve cells (Nagatsu T. Neurosci. Res. 1997 Oct.; 29 (2):99-111). Not only salsolinol and salsolinol derivatives have neurotoxic effects, but also norsalsolinol, N-methyl-norsalsolinol and also other tetrahydro chinolines have neurotoxic effects and lead to neurodegeneration (Storch A, Ott S, Hwang Yl, Ortmann R, Hein A, Frenzel S, Matsubara K, Ohta S, Wolf HU, Schwarz J. Biochem. Pharmacol. 2002 Mar. 1; 63(5):909-20; Naoi M, Maruyama W, Dostert P, Hashizume Y. J. Neural Transm. Suppl. 1997; 50:89-105; Antkiewicz-Michaluk L. Pol J. Pharmacol. 2002 November-December; 54(6):567-72).

### EXAMPLE 6

### Diagnostic importance of 3-CNSa and 3-CNSb and therapeutic consequences

By means of determination methods for 3-CNSa and 3-CNSb in the blood plasma of Parkinson patients treated with L-dopa, those patients can be identified, who have high 3-CNSa and 3-CNSb levels with the aim of the reduction of these neurotoxic salsolinol derivatives.

The aim of the therapy modification is the reduction of the formaldehyde availability in the body by following exemplary measures:

a) Delivering formaldehyde scavengers: due to the electrophilic property of the formaldehyde, it reacts with a variety of compounds such as glutathione, certain proteins, nucleic acids, folic acid and others.

b) Carrying-out an antibiotic therapy with, e.g., rifaximin for the eradication of the formaldehyde producing microbiota. By means of the method according to the invention, the success of this therapy can be monitored and controlled, since with successful antibiotic therapy, the amount of analyte should decrease. Analog considerations apply for an antimycotic therapy.

c) In case that the increased formaldehyde production results from a SIFO (e.g., sandida), a rifaximin treatment would not be reasonable. Here, an antimycotic therapy should be considered. By means of the method according to the invention, the success of this therapy, too, can be monitored and controlled, since with successful antimycotic therapy, the amount of analyte should decrease.

### EXAMPLE 7

### Development of Alpha-Methyl-Dopa from Carbidopa and Condensation Products of Alpha-Methyl-Dopa with Formaldehyde

The inhibitor of the aromatic amino acid decarboxylase, carbidopa, is administered to Parkinson patients in dosages having a fixed relation to the L-dopa dose of 1:4, together with L-dopa (product example Carbidopa® Duodopa®, Rytary®).

By the analysis of patients treated with carbidopa, it could be shown for the first time that signals appear in the HPLC chromatogram that are identical to alpha-methyl-dopa.

Alpha-methyl-dopa is antihypertensive in two ways:

a) By competitive inhibition of the aromatic amino acid decarboxylase, the development of dopamine, noradrenaline and adrenaline is inhibited, and thus the blood pressure in the periphery is reduced.

b) Alpha-methyl-dopa is converted by the dopamine-β-hydroxylase to alpha-methyl-noradrenaline, an alpha2-receptor agonist that is reducing the blood pressure through a central-nervous attack.

### EXAMPLE 8

### Condensation Products of Droxidopa (Dihydroxyphenylserine) with Formaldehyde

Droxidopa is a prodrug of the noradrenaline and adrenaline and is used for the treatment of the neurogenic orthostatic hypotension and diseases that are associated with a central nervous noradrenaline deficiency.

### EXAMPLE 9

### Use of the Method for Finding Formaldehyde Scavengers

By means of the method according to the invention, prospective formaldehyde scavengers can be investigated as to whether they are effective in the organism. For this purpose, a prospective formaldehyde scavenger is administered to test persons in a given dosage, and after a defined period of time, the effect on the amount of analyte in a removed body fluid or in removed cells is determined. Different prospective formaldehyde scavengers are compared to each other with regard to their effects by that the respective amounts of analyte with the same dosage and after the same period of time are compared to each other.

### EXAMPLE 10

### Use of the method for monitoring dietetic measures

Dietetic measures may lead, due to the selectively delivered foodstuffs, e.g., avoiding sugar, to a reduction of formaldehyde/glyoxal in the intestinal tract. By means of the method according to the invention, it can be monitored, whether a diet plan will lead to such a reduction of formaldehyde/glyoxal in the intestinal tract and whether a modification of the diet plan potentially carried out in response thereto will cause an improvement, i.e. reduction of the formaldehyde/glyoxal.

Analog considerations apply with regard to monitoring and possibly modifying the diet habits.

### EXAMPLE 11

### Determination of Free and Conjugated (Thermally/Enzymatically) L-Dopa, Catecholamines and their Formaldehyde Condensation Products in the Urine

The biogenic amines dopamine, noradrenaline, adrenaline and serotonine and their precursors L-dopa and 5-hydroxytryptophane are bound from the biological matrix to a strong cation exchanger and are then sequentially desorbed. The condensation products of the compounds with formaldehyde behave herein same as the educts and are thus also accessible via the selected sample preparation.

### 1. Materials

1.1 Substances, solvents and solutions:
Aluminum oxide
Methanol gradient grades (VWR Prolabo)
Hydrochloric acid 37% (Merck)
Phosphoric acid 85% (Merck)
Sodium hydroxide (Merck)
Ammonium acetate (Roth)
Ethylendiamine-tetraacetic acid disodium salt-dihydrate
(Roth)
TRIS (Roth)
DOWEX 50W-X4 cation exchanger resin
Sodium disulfite (Merck)
Demineralized water (MilliQMillipore)
Mercaptoethanol (Fluka Chemie AG)
β-Glucuronidase/arylsulfatase from Helix pomatia stabilized
aqueous solution(Merck)
Ammonium acetate (Rhom)
L-dopa (EGA-Chemie)
Alpha-methyldopa (Sigma)
3-CNSa (3-carboxynorsalcolinol, Analyticon)
Isoproterenol (Sigma)
Dopamine HCl (Sigma)
Salsolinol
Norsalsolinol (made by ourselves)
   Preparation of the solutions:
a: Standard solutions:
a.1 L-dopa standard solution
a.2 CNSa standard solution (3-carboxynorsalsolinol)
a.3 Alpha-methyl-dopa standard solution
a.4 Isoproterenol standard solution
a.5 Norsalsolinol standard solution (norsalsolinol was made by ourselves from a mixture of dopamine and a formaldehyde solution; standard solution contains approx. 0.45 mg/ml norsalsolinol)
a.6 Dopamine standard solution
a.7 Salsolinol standard solution

The standard solutions a.1-a.4 are prepared as follows. 10 mg of the above substances are given into a 10-ml volumetric flask and filled up with 0.01M HCl to 10 ml. The ready solution contains 1 mg/ml.

These solutions are kept in the refrigerator or aliquoted and stored at −20° C. Before each test, a standard is measured and factors are determined in relation to the internal standard.
b: Further solutions used
0.2M EDTA solution in water
2M HCl
2M NaOH/6M NaOH
6M HCl
0.2M ammonium acetate buffer pH 5.5
0.25% sodium disulfite solution (freshly prepared before each test)
Ethanol: 6M HCl (1:1)
0.5M acetic acid
0.01M HCl
2M ammonium acetate buffer pH 5.5
β-Glucuronidase/sulfatase dilution (1:2 with water) (freshly prepared before each test)
0.2M EDTA solution
2M Tris/HC1 buffer pH 8.6
2M Tris/HC1 buffer pH 9.6
Mercaptoethanol solution (28 p1/10 ml water) (freshly prepared before each test). The solutions mentioned under b may be at RT.

### 1.2 Equipment

Deep cooler (-20 ° C.)
Centrifuge 3200 Eppendorf
Centrifuge 5430R (Eppendorf)
pH meter (Schott)
Whirl Mix (Labin Duo Press to Mix)
Rotation vacuum concentrator RVC 2-25 CD Plus (Christ)
Thermomixer (Eppendorf)
Pipettes, multipipettes (Eppendorf)
Columns with approx. 0.6 cm diameter (for adding the ion exchanger)
Eppis (2 ml/5 ml)
RotiLabo reaction vessels 2 ml (Roth)
Pipette tips (Eppendorf)
Microinserts clear glass for bottles with narrow opening Vol.: 0.1 ml (neoLab)
Thread bottle effective volume: 1 ml (Klaus Ziemer GmbH)
Automatic sampler (Perkin Elmer Series 200 Autosampler)
Integrator: Merck HITACHI D-2500 Chromato-Integrator
Pre-column: YMC Europe GmbH Triart C18 5/pack 10×4.0 m 1. D
S 5 μm, 12 nm TA 12505-0104GC No-152746
Column: YMC HPLC Column, size: 250×4.6 mm i.d., S-5 μm,
12 nm, TA12505-2546WT, No. 0425077073
Detector: Esa Coulochem II (FA0502)
Degasser: Erma ERC 3215 alpha
Pump: RHEOS 4000
Pulsation damper (Shodex Damper)

### 2. Method

2.1 Detection of conjugated and free acids/amines in the urine, the method is carried out in principle as follows.

A. Preparation of the columns with cation exchanger takes place by that the 0.6 cm columns are filled up with DOWEX 50W-X4 (1 cm filling height) and conditioned or regenerated as follows:

Washing with 1×6 ml (2M NaOH incl. 1% EDTA), 2×6 ml of water, 1×6 ml of 2M HCl and 2×6 ml of water.

B. Preparation of the urines for thermal hydrolysis or for determining free analytes (in the 5 ml Eppendorf vial) occurs with this reaction mixture (thermal hydrolysis): Presenting 0.5 ml of urine, 0.5 ml of water and 1 ml of 0.2M HCl, and addition to the int. standards (100 ng of isoproterenol, amines, or 50 ng of alpha-methyldopa, acids)

Bl. Cleavage of conjugated acids/amines takes place by means of thermal hydrolysis (only when determining conjugated acids/amines) and incubation of the reaction mixture for 1 h at 95° C. in the agitated water bath. After incubation and cooling of the sample, the sample is given onto the regenerated ion exchanger.

B2. When only the free acids/amines in urine are to be determined, this step of the thermal hydrolysis is skipped, and the above reaction mixture is directly given onto the regenerated ion exchanger.

C. Preparation of the urines (in the 5 ml Eppendorf vial) for the enzymatic hydrolysis takes place with the following reaction mixture: 0.25 ml of urine, 0.25 ml of water, 0.1 ml of 2M ammonium acetate buffer pH 5.5, 0.05 ml of mercaptoethanol solution (28 p1/10 ml), and 0.02 ml of β-glucuronidase-sulfatase solution (1:2 dilution with water) are mixed with each other.

C1. Determination of conjugated acids/amines takes place by means of enzymatic hydrolysis (only with determination of conjugated acids/amines) by incubation of the reaction mixture for 1 h at 37° C. in the agitated water bath. After the incubation at 37° C., the reaction mixture is acidified as follows. Addition of 200 μl of 2M HCl and 2 ml of water to the incubation reaction mixture. Only then takes place the addition of the int. standards (100 ng of isoproterenol, amines, or 50 ng of alpha-methyldopa, acids). Then the sample is given onto the regenerated ion exchanger.

D. Ion exchange and elution take place by that the total mixture is given onto the prepared ion exchanger. After passage of the samples, the column is washed 3 times with 4 ml of water. The elution of the acids from the ion exchanger takes place with 1×1.5 ml of 0.2M ammonium acetate buffer pH 5.5 (discard fraction) and 1×3.0 ml 0.2M of ammonium acetate buffer pH 5.5 (use fraction for Al₂O₃ binding). The ion exchanger column is washed once again with 4 ml of water. The elution of the amines from the ion exchanger takes place with 1×2 ml (ethanol: 6M HCl, 1+1)

E. Aluminum oxide binding (acids) takes place as follows. Preparation: 20 mg of aluminum oxide are given into a 2 ml Eppi, and the reaction mixture is added in the following sequence: take 1 ml from the 3 ml of ammonium acetate acid eluate, add 20 μl (0.25% sodium disulfite in water), 20 μl (0.1M EDTA) and 200 μl (2M Tris buffer pH 9.6). The aluminum oxide is washed 3× with 1 ml of 0.001M EDTA solution.

Aluminum oxide binding (amines) takes place as follows. Preparation: 20 mg of aluminum oxide are given into a 5 ml Eppi, and the reaction mixture is added in the following sequence: take 0.5 ml from the 2 ml of HC1:ethanol amine eluate, add 0.5 ml of water, 20 μl of 0.25% sodium disulfite in water, 100 μl of 0.2M EDTA, 500 μl (2M Tris buffer pH 8.6) and 500 μl of 2M NaOH. The total reaction mixture is mixed for 10 min/2000 rpm at 10 ° C. in the thermomixer. The aluminum oxide is washed 3x with 1 ml of 0.001M EDTA solution.

Desorption from the aluminum oxide takes place by that 500 μl of 0.5M acetic acid are given onto the aluminum oxide, mixed for 10 min in the thermomixer at 21° C. and 2000 rpm and concentrated by that 0.5 ml of supernatant (desorbate) are removed and transferred into a 2 ml Eppendorf vial and brought into the vacuum concentrator at 35° C. for drying (approx. 3 h)

The measurement takes place by that a sample is received in 500 μl of mobile phase, vortexed for 30 sec and transferred into a microvial. 5 μl-10 μl are employed in the HPLC.

F: HPLC conditions are as follows. Column: YMC HPLC Column, size: 250×4, 6 mm i.d., S-5 min, 12 nm, TA12S05-2546WT, No. 0425077073; pre-column: YMC Europe GmbH, TriartC18 5/pack, 10×4.0 mml. D S-5 μm, 12 nm, TA 12505-0104GC, No-152746; rinsing liquid: 50% methanol in 0.005M HCl; flow: 0.8 ml/min.; temperature: room temperature; column pressure: 120 bars; mobile phase: 20% Na2HPO4*2H₂₀ pH 2.5, 0.1% (0.2M EDTA in water; the mobile phase is filtered under vacuum through a GXWP 04700 0.22 μm filter); setting of the esa Coulochem II Detector: guard cell: +350 mV, measurement cell: +250 mV, current: 500 nA.

2.2 Calculation

Calculation of the concentration of the analyte takes place in a common way by the peak area relation of the added amount of internal standard to the analyte.

1. A method for the qualitative, semi-quantitative, or quantitative determination of aldehyde-producing microorganisms in the intestinal tract, in particular of the small intestine, of a human on a body fluid removed from a human and/or in cells taken from a human, wherein on the body fluid and/or the cells a qualitative, semi-quantitative, or quantitative chemical, physical, or physico-chemical determination of a condensation product of a Pictet-Spengler reaction between a carbonyl compound, in particular an aldehyde, preferably formaldehyde, and an amine as an analyte is carried out.

2. A method for the qualitative, semi-quantitative, or quantitative determination of a condensation product of a Pictet-Spengler reaction between a carbonyl compound, in particular an aldehyde, preferably formaldehyde, and an amine in a body fluid removed from a human and/or in cells taken from a human, wherein on the body fluid and/or the cells a qualitative, semi-quantitative, or quantitative chemical, physical, or physico-chemical determination of the condensation product as an analyte is carried out.

3. The method according to claim 1, wherein the microorganisms to be determined are carbonyl compounds-producing, preferably aldehyde-producing, in particular formaldehyde-producing.

4. The method according to claim 1, wherein the body fluid is selected from the group consisting of urine, full blood, blood plasma, serum, and liquor, and wherein the cells are selected from the group consisting of erythrocytes, leukocytes, and thrombocytes.

5. The method according to claim 1, wherein the amine is a catecholamine with a primary amine group, in particular dopamine or L-dopa.

6. The method according to claim 1, wherein the amine is a substance of formula I: [chemical structure]
wherein R₁ is selected from —CH₃, —CH₂OH, —COOH, —COOCH₃, —COOC2H₅, —CONH₂, —OH, —OCH₃, —OC₂H₅,
wherein R₂, R₃ and R₄, each independently of the other, is selected from —H, —OH, —CH₃, —C₂H₅, —OCH₃, —OC₂H₅, wherein R₁ and R₂, alternatively to the above definitions, may together also be ═O,
wherein R₅ is selected from —H, —CH₃ and —C₂H₅,
wherein R₆ is —H or —OH,
wherein R₇ is —H or —OH,
wherein R₈ and R₉, each independently of the other, are selected from —H, —OH, —OCH₃ and —OC₂H₅, and
wherein R₁₀ is —H or —OH.

7. The method according to claim 1, wherein the condensation product is 6,7-dihydroxy-3-carboxy-1,2,3,4-tetrahydroisoquinoline or 7,8-dihydroxy-3-carboxy-1,2,3,4-tetrahydroisoquinoline.

8. 7,8-Dihydroxy-1,2,3,4-tetrahydroisoquinoline or a 7,8-dihydroxy-1,2,3,4-tetrahydroisoquinoline derivative, wherein in the 7,8-dihydroxy-1,2,3,4-tetrahydroisoquinoline derivative
—H in position 3 may be replaced by —CH₃, —CH₂OH, —COOH, —COOCH₃, —COOC2H₅, —CONH₂, —OH, —OCH₃, —OC₂H₅, —OH, —OCH₃, or —OC₂H₅, one —H or both —H in position 4, independently of the other, identically or differently, may be replaced by —OH, —CH₃, —C₂H₅, —OCH₃, or —OC₂H₅, and one —H or both —H in position 5 or 6, independently of the other, identically or differently, may be replaced by —OH.

9. Use of 7,8-dihydroxy-1,2,3,4-tetrahydroisoquinoline or of a 7,8-dihydroxy-1,2,3,4-tetrahydroisoquinoline derivative according to claim 8 or of 6,7-dihydroxy-3-carboxy-1,2,3,4-tetrahydroisoquinoline or of a 6,7-dihydroxy-3-carboxy-1,2,3,4-tetrahydroisoquinoline derivative, wherein
—H in position 3 may be replaced by —CH₃, —CH₂OH, —COOH, -COOCH₃, —COOC₂H₅, —CONH₂, —OH, —OCH₃, —OC₂H₅, —OH, —OCH₃, or —OC₂H₅ one —H or both —H in position 4, independently of the other, identically or differently, may be replaced by —OH, —CH₃, —C₂H₅, —OCH₃, or —OC2H₅ —H in position 7 or 8, independently of the other, identically or differently, may be replaced by —OH,
as an analyte in a method according to claim 1.
